(19) United States
(12) Patent Application Publication
(10) Pub. No.: US 2018/0098790 A1
(43) Pub. Date: Apr. 12, 2018

(54) PERINEAL PROTECTION DEVICE AND EXTRACTION BAG, PERINEAL HORSESHOE AND SLEEVE

(71) Applicant: AIR BAG ONE SARL, Luxembourg (LU)

(72) Inventor: Jorge Ernesto ODON, BUENOS AIRES (AR)

(73) Assignee: AIR BAG ONE SARL, Luxembourg (LU)

(21) Appl. No.: 15/533,601

(22) PCT Filed: Jun. 9, 2016
(86) PCT No.: PCT/EP2016/063220

§ 371 Date: Jun. 6, 2017

(30) Foreign Application Priority Data
Jun. 10, 2015 (AR) 20150101828

(51) Int. Cl. A61B 17/42

(52) U.S. Cl. CPC A61B 17/42; A61B 2017/00907

(57) ABSTRACT

A device to protect the perineal and an extraction bag. This device has a perineal horseshoe and an optionally perineal forchette. The perineal horseshoe is a “U-shaped” piece with a wide base. The side opposite to the wide base defines a curved surface. The bag is a sleeve folded on itself, producing two concentric tubes. These tubes formed a pair of traction handles. The perineal horseshoe has at least one guide rail and one of the tubes, whether internal or external, have a guide that works in coordination with the herein mentioned guide rail. The perineal forchette consists of a supportive central axis, a handle, a tab and two articulated arms linked to the central axis. The sleeve can have a pair of additional traction handles in its distal end.

### APPLICATION FIELD

The current invention refers to a perineal protection device that avoids and/or reduces injuries, lacerations or tears, in particular the perineal injuries, lacerations or tears at the moment of crowning of the cephalic pole, to be used with devices that extract elements within a cavity, like a sleeve folded on itself.

### PRIOR ART OF THE INVENTION AND ITS BENEFITS

The perineum is the anatomical area located below the pelvic floor and it consists of a group of soft tissue parts that seal the bottom of the pelvis. This is a delicate area, especially during labor.

During childbirth, the fetus applies pressure over the perineum; pushing it outwards, stretching the muscles of the perineum, and even causing perineal tears. The labor assistant holds the perineum on one hand to counteract the pressure, so it only has one free hand.

These perineal tears have no control over their depth, direction and, especially over length. They rise at the lower part of the muscles of the surface of the vaginal opening and can run directly towards the anus (medium perineal tear), towards the side (lateral perineal tear) or towards an intermediate area (lateral and oblique perineal tear).

Perineal tears are the source of a wide variety of problems, both physical and psychological, in women that had suffered them, especially if the tearing was the medium kind and went on near the anus.

The most important problems that worth mentioning as a consequence of perineal tears are: infections, edemas, hematomas, transitory or permanent dyspareunia (pain during sexual intercourse), underlying abscesses, muscular or nervous retraction, partial or complete urinary incontinence, partial or complete bowel incontinence (both transitory or permanent), anal and vaginal fistula, aggravated hemorrhoids, perineal-vulvar thrombosis, scar endometriosis, anemia, psychological trauma.

An episiotomy was performed to avoid these “uncontrolled” perineal tears during labor. The episiotomy is a surgical incision between de vagina and the anus. It replaces the tearing but is more controlled in regards its direction and length, and it is a straight incision. All these features help the healing process.

In some labors where the vaginal opening is small, the fetus comes in breech position or the fetus has a big head, that opening has not enough space or flexibility for the fetus to go through without harm. Naturally, a perineal tear is produced starting at the vaginal opening. The episiotomy has proven to reduce considerably the consequences produced by perineal tears. It is even practiced when is not necessary.

Patent GB 1127548 of September 1968, states to be a “device to reduce the amount and seriousness of perineal tears during labor.” The device consists of an elastic horn-shaped elastic limb for the back part of the perineum, below and on the sideways of the entrance of the vagina up to the anal opening, with a side attached to the perineum itself. The degree of elasticity increases progressively towards the sides of the device. Elasticity changes may be obtained by altering the properties of the material, but it is ideal to change the density of the device.

Patent WO 2009101186 of August, 2009, states to be a “device to protect the tissue during labor.” One of the objects of invention is a “device to reduce the tissue tension during labor,” which consists of a sheet of flexible material that has a top side and a bottom side, where at least the bottom one, during use, is shaped to be kept attached with the area contiguous to the entrance of the vagina, or a second that consists of an area further the entrance of the vagina, where the sheet is shaped by any of those two areas during use and when in contact with the birth canal walls and the top side is far from the birth canal wall. In one method, the device stuck to the perineum is a mix of irregular surfaces to improve the strength distribution effect.

The application for the patent US 2007/0260163 of November, 2007, states to be a “device and method to prevent perineal tissue damage.” The device consists of a limb to fix the device itself by each anchor, preferably with Velcro, on the buttocks of the woman in labor.

The application for the utility model CN 201208289 of March, 2009, states to be a “protective device for childbirth,” a medical device to apply external force and prevent an anal sphincter injury during labor. It consists of a hard foundation covered by a flexible material with an arched side that matches with the outside of the perineum so that the foundation can bear the force applied evenly and relax the perineal muscles. It has indentations to put the fingers for an easy handling of the device.

The patent U.S. Pat. No. 4,520,807 of June, 1985, claims to be an “obstetrical device and method.” It is a device to minimize and/or eliminate the infection during labor and obstetric interventions. It consists of an adhesive portion with a protective portion underneath. The adhesive portion is stuck below the entrance of the vagina, with the protective portion covering the anus. This way, it prevents the feces from infecting the perineum and the perineal area during labor and obstetric interventions.

### MAIN PURPOSES OF THE CURRENT INVENTION

The main purpose of the current invention is to promote a perineal protection device that prevents injuries, lacerations or perineal tears during labor, by forcing the perineal tissue to remain in its normal position.

Another purpose of the current invention is to simplify the placing of the perineal protection device in position by simply gliding it through guides towards the perineum, where it will remain it optimum position to work.

Another purpose of the current invention is to provide a perineal protection device easy to use.

Another purpose of the current invention is to provide a perineal protection device that is not inserted in the vagina.

Another purpose of the current invention is to provide a flexible and stretchable perineal protection device.

Another purpose of the current invention is to provide a perineal protection device that can bear the pressure of the cephalic pole during crowning to avoid the swelling of the vulva that entail injuries, lacerations or perineal tears.

Another purpose of the current invention is to provide a device that can bear the pressure of the cephalic pole during crowning to help relaxing the perineal tissue.

Another purpose of the current invention is to provide a device that simplifies the opening of the vagina during crowning.

Another goal of the current invention is to provide device to extract elements present inside a cavity by relying on its guides to adjust the perineal protection device in its proper position.

### BRIEF DESCRIPTION OF THE DRAWINGS

In order to make the object of invention more comprehensible, it has been illustrated with simplified figures, embodied as preferred, which act as demonstration:

FIG. 1 is a perspective of a device to extract elements present inside a cavity, according to an initial development model.

FIG. 2 is a cross-sectional view of the concentric tubes, showing the different possible locations for the guides.

FIG. 3 is a second development model of a device to extract elements present inside a cavity.

FIG. 4 is a third development model of a device to extract elements present inside a cavity.

FIG. 5 is a forth development model of a device to extract elements present inside a cavity.

FIG. 6 is a first model of the perineal horseshoe.

FIG. 7 is a second model of the perineal horseshoe.

FIG. 8 is a third model of the perineal horseshoe.

FIG. 9 is a perineal horseshoe with a first model of perineal forchette.

FIG. 10 is a perineal horseshoe with a second model of perineal forchette.

FIG. 11 is the layout of the trigger.

FIG. 12 is a frontal view of the device to extract elements present in the cavities and the perineal protection device in using position.

FIG. 13 is a side view of the device to extract elements present in the cavities and the perineal protection device in using position.

### DETAILED DESCRIPTION OF THE INVENTION

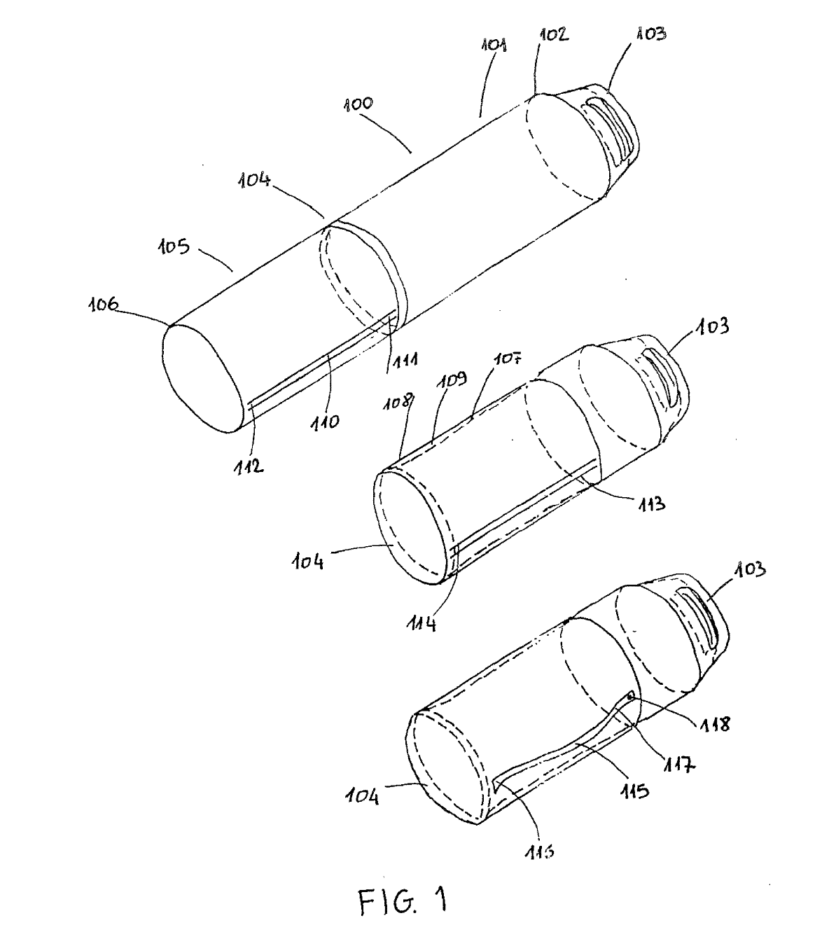

FIG. 1 depicts a device to extract elements present inside a cavity that consists of a sleeve (100) with two open ends of a flexible material, preferably transparent and with a density that allows an easy folding, which is introduced in folded position inside the birth canal, between the cephalic pole of the fetus and the birth canal, adjusting itself to the environment. With the sleeve unfolded, it covers a proximal portion with the first end open (102) that has traction handles (103), a folding portion (104) and a distal portion (105) with the second end open (106). In use, the sleeve is folded on itself in the folding portion, producing two concentric tubes, the proximal portion creating an internal tube (107) and the distal portion creating an external tube (108), with a space (109) in between.

The sleeve prepared for the current invention requires at least one guide (110) connected by its end (111) on the side of the fold and its end (112) on the side of one of the open ends to one of the concentric tubes produced by folding the sleeve. The first chosen method would be to make a longitudinal section on the lower axis in at least one of the concentric tubes in order to create a guide. A second choice would be to create a guide by making two longitudinal sections (113, 114) in at least one of the concentric tubes. A third choice would be to make three longitudinal sections in at least one of the concentric tubes to create the guides. A forth choice would be to form two guides by making three longitudinal sections in at least one of the concentric tubes in order to create two guides. If creating just one guide, it would be better to do it over the lower axis of the sleeve. When creating two guide bands, both should be located near the lower axis of the sleeve, preferably in symmetric position.

At least one guide should be of flexible material, preferably the same material of the sleeve, and it should be slightly tense when pushing the end of the bag. This minor tension should be strong enough for the perineal horseshoe to slide over itself, as it will be seen further on.

With another method, the guide (115) is added on the outside of the sleeve. The end (116) on the side of the fold of the guide is permanently fixed to the sleeve in the folding area, while the opposite end (117), on the side of one of the open ends of the sleeve, can be fixed or removable from the sleeve. In this last case it should be done by attaching it (118) with clasps, hooks, Velcro bands or any similar method to fix the end of the guide to one of the open ends of the sleeve, to be removed afterwards. These fixing elements, with their other side over the tube, should be strong enough to bear the tension on the tube while pulling by the traction handles.

FIG. 2 depicts a cross section of one of the concentric tubes, showing the different possible locations for the guides. The internal tube (201), the external tube (202) and the dots (203) show the different possible locations for the guides. The dots indicate either the longitudinal section over the tube when the guide is done over the tube itself or the circumference arc over the tube of the ends of the guide when it is added outside the sleeve.

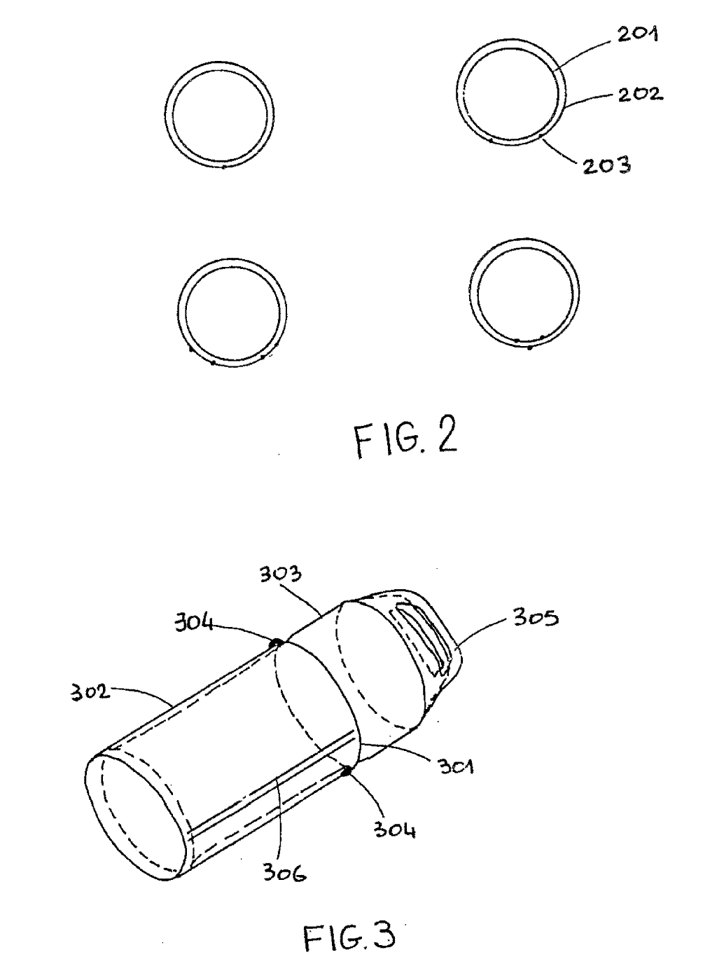

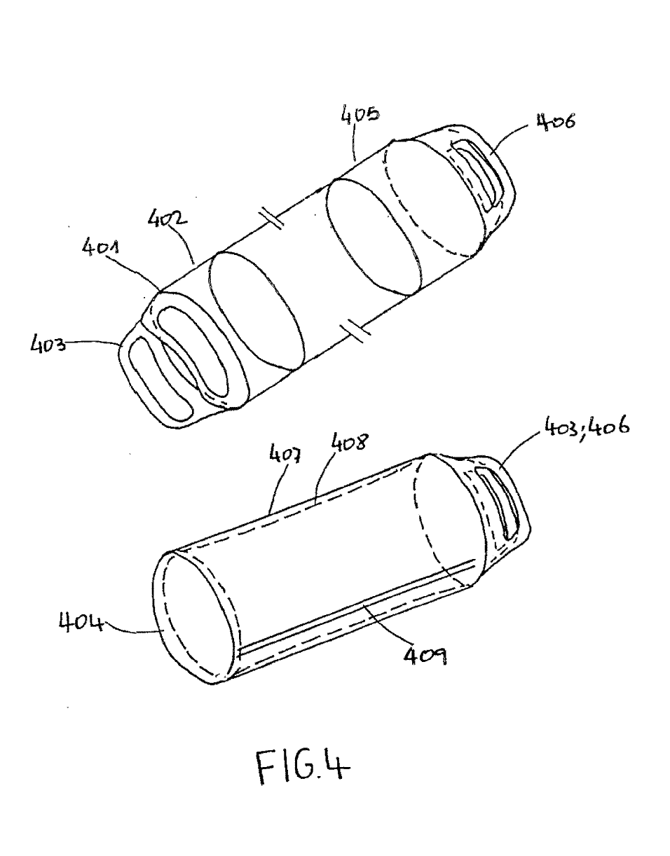

FIG. 3 depicts the sleeve adapted for the current invention in a first development. The open end (301) of the external tube (302) is fixed to the internal tube (303) by at least one fastening (304), which can be fixed or removable like clasps, hooks, Velcro bands or any similar method. This purpose of this fastening is that, while pulling the traction handles (305) fixed on the internal tube, the external tube and at least one guide (306) located over the external tube are also tensed. In this particular case, the guide can be done by cutting a longitudinal section in the lower end of the external tube. These fastenings, with their over the tube if removable, should be strong enough to bear the tension on the tube while pulling by the traction handles. The fastenings should leave enough space between the internal tube and the external tube as to allow the access of the different elements used during labor, as the tools used to grab the cephalic pole.

FIG. 4 depicts the manga adapted for the current invention in a third development. The open end (401) of the distal portion (402) of the sleeve also has a pair of traction handles (403). When folding this distal portion over the folding portion (404), that shares the center with the proximal portion (405) and has its own traction handles (406), both pairs of traction handles are aligned so that they can be pulled simultaneously, the ones fixed (406) to the external tube and the ones fixed (403) to the external tube. This way, both the external (407) and the internal (408) tubes and at least one guide (409) are tensed. In this particular case, the guide can be done by making a longitudinal section in lower end of the inferior tube.

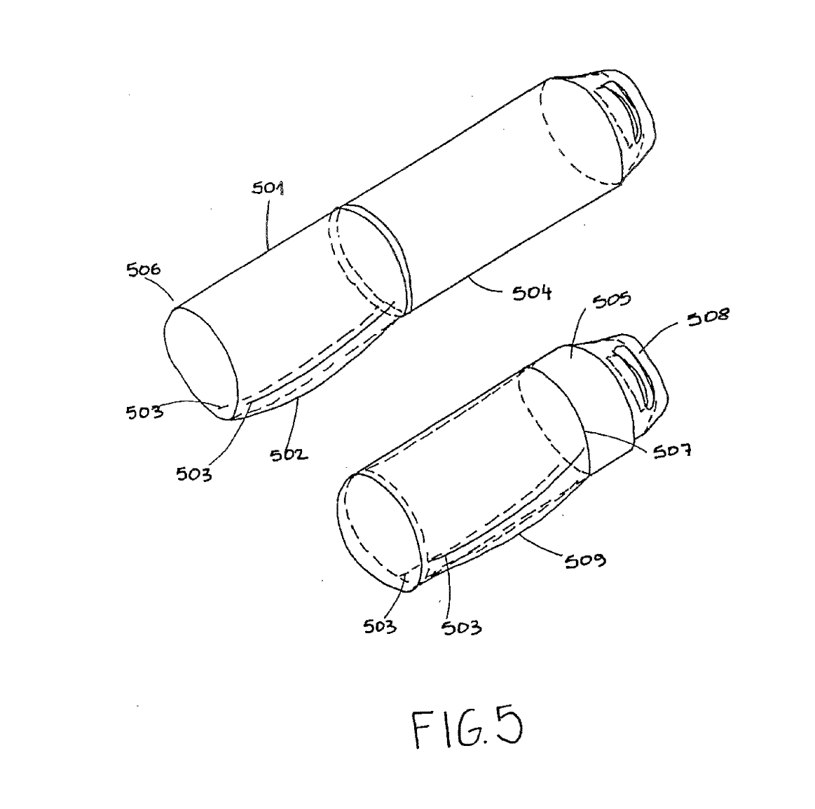

FIG. 5 depicts a sleeve adapted for the current invention in a forth development. The external tube (501) shows at least one cut (502), preferably two more longitudinal cuts (503) on the sides to form a guide. In at least one cut, the inferior portion (504) of the internal tube (505) is left exposed. In this method, the open end (506) of the external tube is linked to the internal tube by all of its edges (507). By pulling the traction handles (508), both the concentric tube and at least one guide are stretched, which in this version is located over the external tube.

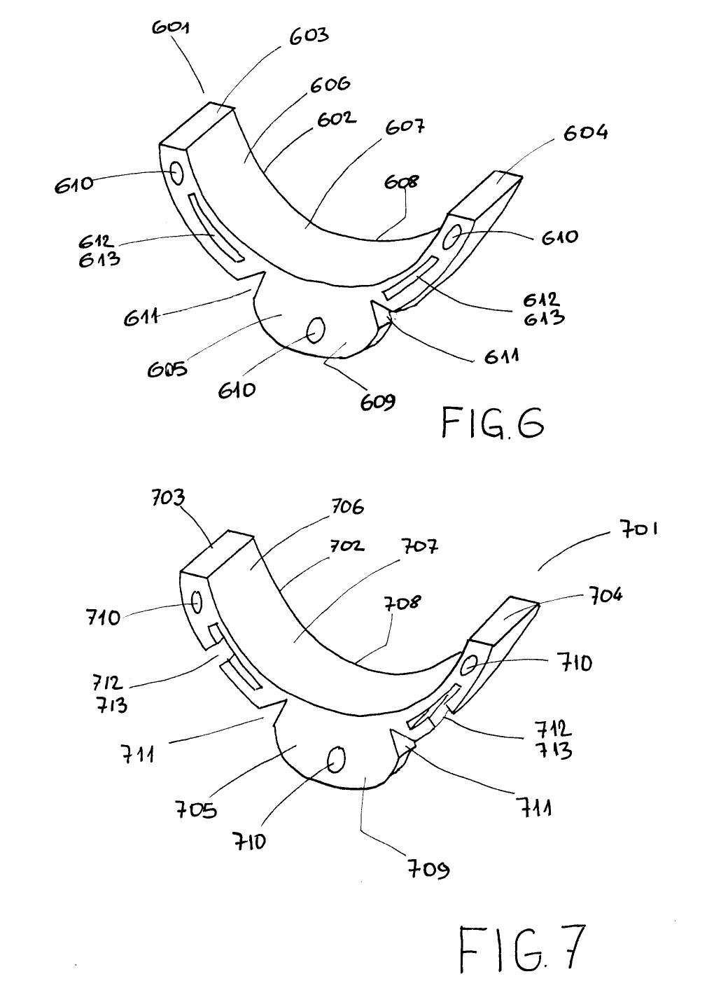

FIG. 6 depicts a perineal protection device in its first development. It consists of a perineal horseshoe (601) and an optional perineal forchette. The perineal horseshoe is a “U-shaped” piece (602) with two arms (603, 604) and a wide base (605). The side opposite to the wide base has a circumference arc (607), preferably with a ratio inferior to one of the sleeve when open. The perineal horseshoe has two perpendicular levels, one proximal side (608) and one distal (609). The proximal side has joints (610) to attach it to the perineal forchette. The perineal horseshoe has curved borders and such curvatures are soft enough to prevent injuries in the perineal tissue. The perineal horseshoe is made of flexible material that can be bended obliquely to fit the different measures of the vaginal opening. Optionally, it can come with compliance tools, which can be slots (611) or at least one joint to articulate obliquely both arms to the wide base. It has at least one guide rail (612).

The perineal horseshoe has guide rails that are sharp through holes (613) and where the guides are placed for the perineal horseshoe to move over. In this development, the guides should be the kind that can be detached from the bag. The guides are detached from the bag, threaded into the sharp through holes and attached to the end of the guide to the bag.

FIG. 7 depicts a perineal protection device in a second development. It consists of a perineal horseshoe (701) and an optional perineal forchette. The perineal horseshoe is a “U-shaped” piece with two arms (703, 704) and a wide base (705), which has a circumference arc (707), preferably with a ratio inferior to one of the sleeve when open. The perineal horseshoe has two perpendicular levels, one proximal side (708) and one distal (709). The distal side has joints (710) to attach it to the perineal forchette. The perineal horseshoe has curved borders, soft enough to prevent injuries in the perineal tissue. The perineal horseshoe is made of flexible material that can be bended obliquely to fit the different measures of the vaginal opening. Optionally, it can come with compliance tools, which can be slots (711) or at least one joint to articulate obliquely both arms to the wide base. It has at least one guide rail (712). The perineal horseshoe has guide rails that are sharp through holes (713) and where the guides are placed/removed and where the perineal horseshoe can move over. In this development, the guides are added sideway through the sharp hole until it completely covers the inside of the sharp through holes. It is clearly noticed that, in this case, the guides' ends can be fixed to the bag.

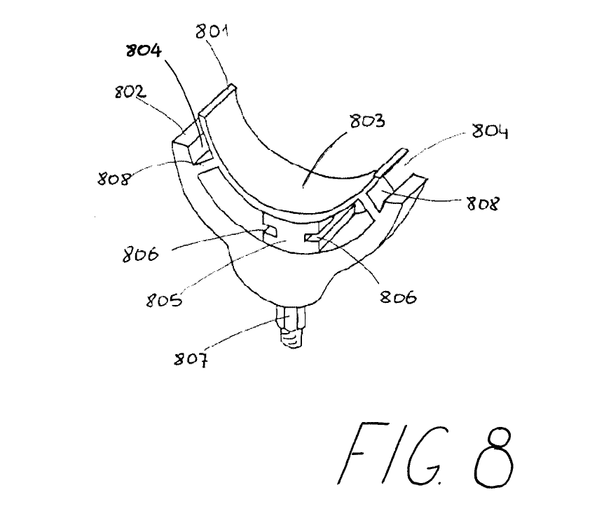

FIG. 8 depicts a perineal protection device in a third development. The perineal horseshoe consists of two halves (801, 802) linked by their center (803), leaving space (804) between them. The linking element (805), preferably square and optionally fixed to any of the halves or independent, has in its two faces a longitudinal opening (806) that will work as guide rails. The guide of the sleeve is put inside these openings, allowing the perineal horseshoe to slide over them. The connection between the perineal horseshoe halves can be fixed or removable, and it can be done by joints, screws (807), wing nuts or similar ones. In this development, a knob (808) can be added between both halves to firmly link both ends so that one lateral plane can drag the other while sliding.

The perineal protection device of any previously mentioned development can include a perineal forchette.

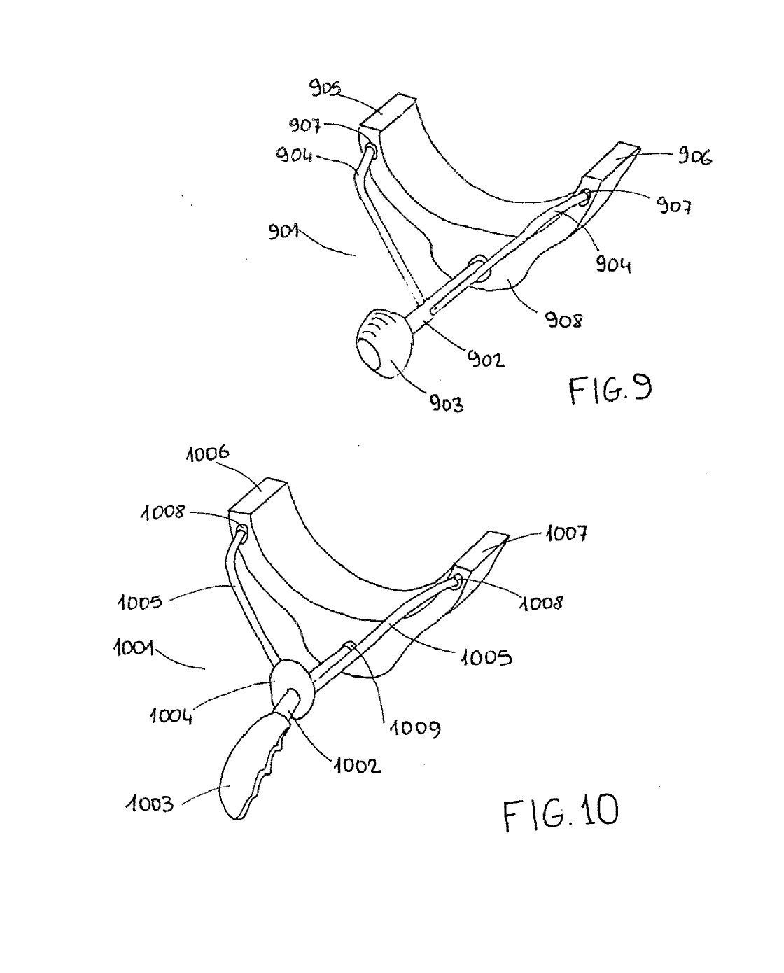

FIG. 9 depicts a perineal protection device where the perineal forchette is a knob (901). The tope consists of a supportive central axis (902), a handle (903) and two articulated arms (904) linked to the central axis. Each distal end of the perineal horseshoe (905, 906) has a joint (907) to fit the end of both articulated arms (904). The end of the supportive central axis connects with the wide base through a joint (908). The current shaping shows that by keeping the perineal horseshoe over the perineum of the woman in labor, the perineal tissue is protected, preventing a swelling in the perineum and the vulva. If the perineal forchette is pushed towards the bag and the perineum, the articulated arms will open, pushing the perineal horseshoe's arm to split up and also pushing the bag to open, opening the vagina.

FIG. 10 depicts a perineal protection device where the perineal forchette is a haft (1001). The haft consists of a supportive central axis (1002), a handle (1003), a flap (1004) and two articulated arms (1005) linked to the central axis. It is preferable that the supportive central axis is tilted away the bag. The flap will hold the thumb when handling the perineal horseshoe. Each distal end of the perineal horseshoe (1006, 1007) has a joint (1008) to fit the end of both articulated arms (1005). The supportive central axis connects with the wide base through a joint (1009). The current shaping shows that by keeping the perineal horseshoe over the perineum of the woman in labor, the perineal tissue is protected, preventing a swelling in the perineum and the vulva. If the perineal forchette is pushed towards the bag and the perineum, the articulated arms will open, pushing the perineal horseshoe's arm to split up and also pushing the bag to open, opening the vagina.

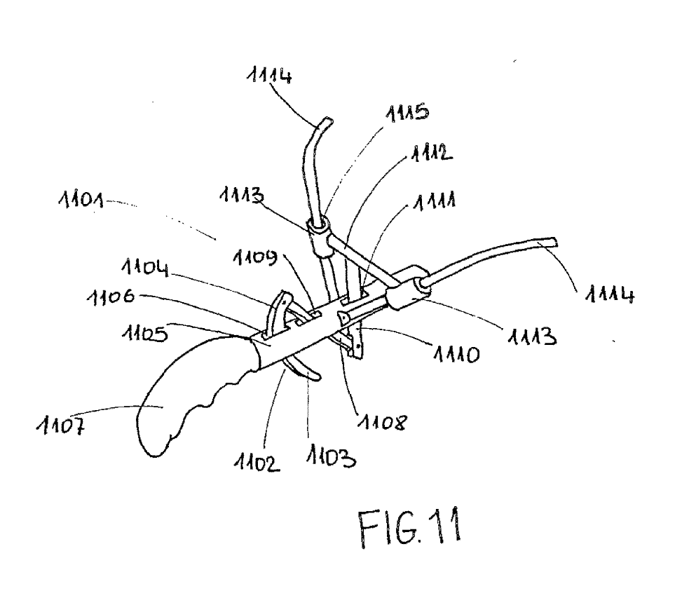

FIG. 11 depicts the layout of the trigger (1101) to separate the articulated arms from the perineal forchette. This helps to open the vagina without releasing the perineum. By separating the articulated arms, the bag attached to them also opens, either the internal or external bag. With traction, the bag separates the outer labia. The layout of the trigger consists of a trigger (1102) with a command section (1103) and an operative section (1104) that goes through the supportive central axis (1105) in the first opening (1106), the pivot point between both sections and aligned with the first opening. It is preferable that the trigger is located in the middle of the supportive central axis close to the haft (1107). The distal end of the operative section is linked from a first bolt to a pushrod (1108) goes through the supportive central axis by a second opening (1109). The second end of the pushrod is connected with a second bolt by the lower end of the pushrod (1110), which also goes through central axis by a third opening (1111). The opposite end of the pushrod is firmly attached to a crosspiece (1112) that has on each end a reducing bushing (1113). These bushings have the same inclination as the articulated arms (1114), leaving a clearance (1115) with the opening of the arms, which is big enough to push the crosspiece and the bushings downwards and let the articulated arms to open.

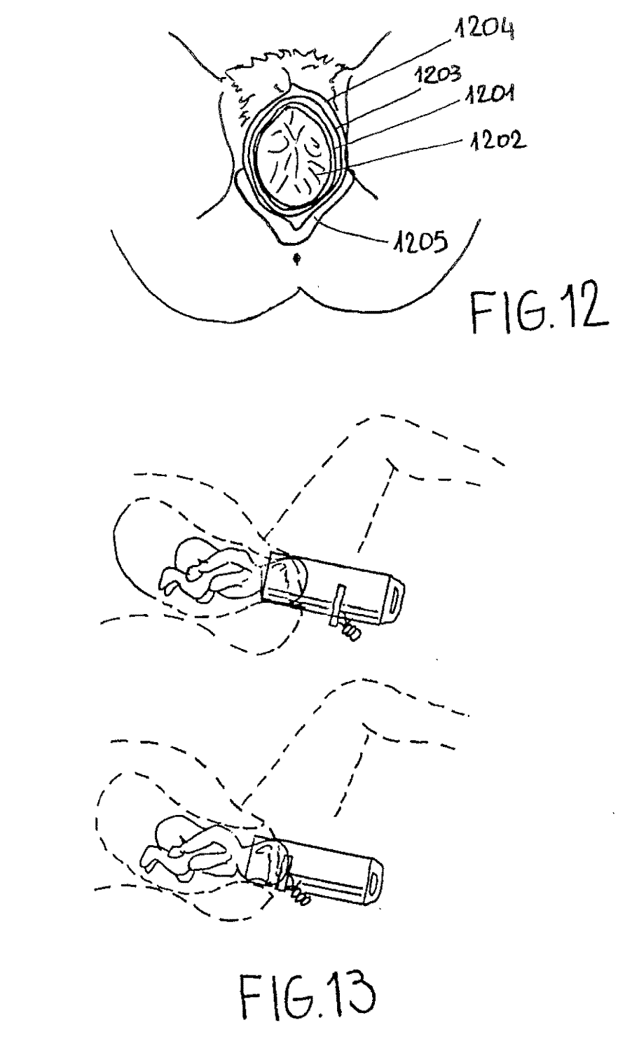

FIG. 12 depicts a frontal view of the complete extraction device ready to use. It is noticed that the bag is located inside the birth canal with the internal tube (1201) in contact with the fetus' head (1202), and the external tube (1203) in contact with the vaginal walls (1204). The perineal horseshoe (1205) is leaned over the perineal tissue, protecting the perineum and the vulva.

FIG. 13 depicts a side view of the complete extraction device ready to use.

For its use, the obstetrician places the bag in position waiting for a contraction to tense the part of the bag where one of the guides is located to slide the perineal horseshoe over it until the knob is located in the perineal tissue. It is easy to operate because the perineal horseshoe linked to the bag by at least one guide automatically positions the perineal horseshoe in its right place, no adjustment needed. It cannot be misplaced or out of place. The obstetrician just has to put the necessary pressure over the perineal horseshoe to counterpart the pressure of the cephalic pole. Operating the perineal horseshoe does not interfere with the operation of the bag, which consists of pulling the traction handles to push the fetus outside the birth canal.

If it is necessary to separate the vagina walls, the obstetrician has to push the handle against the perineum of the woman in labor. Then, he has to bend the command handle towards the bag so to open the arms, pushing the arms of the “U” to open. These arms laterally drag the bag to which they are linked, opening the vagina walls. In one development, the command handle has a trigger or a similar system that helps splitting the arms by adding pressure, opening the arms of the perineal horseshoe.

1-13. (canceled)

14. A perineal protection device and an extraction bag,
wherein the device comprises a perineal horseshoe and an optional perineal forchette,
wherein the perineal horseshoe is a “U-shaped” piece with two arms and a wide base;
wherein the side opposite to the wide base defines a curved surface, and
the extraction bag comprises a sleeve folded on itself creating concentric external and internal tubes, wherein the internal tube has a pair of traction handles on its proximal end;
wherein perineal horseshoe has at least one guide rail and one of the tubes, internal or external, has one guide that works in coordination with the aforementioned guide rail.

15. The perineal protection device from claim 14, wherein the guide rail is a sharp close through hole.

16. The perineal protection device from claim 14, wherein the guide rail is an open through hole.

17. The perineal protection device from claim 14, wherein the perineal forchette comprises a supportive central axis, a handle, a lift tab and two articulated arms linked to the central axis.

18. The perineal protection device from claim 17, wherein the central axis is tilted away from the central axis of the tubes.

19. The perineal protection device from claim 17, wherein the handle further has a trigger layout, comprising a trigger with a command section and an operative section that goes through the supportive central axis in a first opening, being the pivot point between both sections and aligned with the first opening, and wherein the distal end of the operative section is linked from a first bolt to a pushrod that goes through the supportive central axis by a second opening, and wherein the second end of the pushrod is connected with a second bolt by the lower end of the pushrod, which also goes through the central axis by a third opening, being the opposite end of the pushrod firmly attached to a crosspiece that has on each end a reducing bushing that sliding fits in each articulated arm.

20. The extraction bag from claim 14, wherein the first end open of the distal portion has traction handles, which while folding, matches with the traction handles from the internal tube.

21. The extraction bag from claim 14, wherein the open end of the external tube is fixed to the internal tube in at least one fastening.

22. The extraction bag from claim 21, wherein at least one fastening of the attachment from the external tube with the internal can be fixed or removable.

23. The extraction bag from claim 14, wherein at least one of the guides is made on the external tube.

24. The extraction bag from claim 14, wherein at least one of the guides is added externally to the external side of the external tube.

25. The extraction bag from claim 24, wherein at least one of the ends of the guide is removable attached to the external side of the external tube.

26. The extraction bag from claim 25, wherein the fastenings of at least of the ends of the guides is a clasps, a hook, Velcro bands or any similar method.
